(19) United States
(12) Patent Application Publication
(10) Pub. No.: US 2018/0016622 A1
(43) Pub. Date: Jan. 18, 2018

(54) SYSTEMS, METHODS, AND KITS FOR AMPLIFYING OR CLONING WITHIN DROPLETS

(71) Applicant: President and Fellows of Harvard College, Cambridge, MA (US)

(72) Inventors: David A. Weitz, Bolton, MA (US); John Heyman, Somerville, MA (US); Huidan Zhang, Cambridge, MA (US); Linas Mazutis, Vilnius (LT)

(73) Assignee: President and Fellows of Harvard College, Cambridge, MA (US)

(21) Appl. No.: 15/544,942

(22) PCT Filed: Jan. 22, 2016
(86) PCT No.: PCT/US16/14531

§ 371 Date: Jul. 20, 2017

Related U.S. Application Data
(60) Provisional application No. 62/106,982, filed on Jan. 23, 2015.

(51) Int. Cl. C12Q 1/68; C12N 15/10; B01L 3/00; B01L 7/00

(52) U.S. Cl. CPC C12Q 1/686; B01L 7/52; C12Q 1/6806; C12N 15/1075; C12N 15/1065; B01L 3/502784; C12Q 1/6848; B01L 2200/0673; B01L 2300/0829; B01L 2300/0864

(57) ABSTRACT

The present invention generally relates to droplet-based microfluidic devices, including systems, methods, and kits for amplifying or cloning within droplets. In some embodiments, the present invention is generally directed to systems, methods, or kits for amplifying a plurality of nucleic acids, e.g., without substantially selectively amplifying some nucleic acids over others. The nucleic acids may be contained within the droplets. In addition, in some embodiments, a plurality of microfluidic droplet containing a species of interest, such as a nucleic acid, may be mixed with microfluidic droplets free of the species, then pipetted or otherwise transferred such that, on average, a predetermined number of droplets containing species of interest is transferred.

### RELATED APPLICATIONS

This application claims the benefit of U.S. Provisional Patent Application Ser. No. 62/106,981, filed Jan. 23, 2015, entitled “Systems, Methods, and Kits for Amplifying or Cloning Within Droplets,” by Weitz, et al., incorporated herein by reference in its entirety.

### FIELD

The present invention generally relates to droplet-based microfluidic devices, including systems, methods, and kits for amplifying or cloning within droplets.

### BACKGROUND

A variety of techniques exist for producing fluidic droplets within a microfluidic system, such as those disclosed in Int. Pat. Pub. Nos. WO 2004/091763, WO 2004/002627, WO 2006/096571, WO 2005/021151, WO 2010/033200, and WO 2011/056546, each incorporated herein by reference in its entirety. In some cases, relatively large numbers of droplets may be produced, and often at relatively high speeds, e.g., the droplets may be produced at rates of least about 10 droplets per second. The droplets may also contain a variety of species therein.

### SUMMARY

The present invention generally relates to droplet-based microfluidic devices, including systems, methods, and kits for amplifying or cloning within droplets. The subject matter of the present invention involves, in some cases, interrelated products, alternative solutions to a particular problem, and/or a plurality of different uses of one or more systems and/or articles.

In one aspect, the present invention is generally directed to a method. In one set of embodiments, the method includes acts of fragmenting a nucleic acid to produce nucleic acid fragments, containing at least some of the nucleic acid fragments in a plurality of microfluidic droplets, and amplifying at least some of the nucleic acid fragments contained within the microfluidic droplets.

In accordance with another set of embodiments, the method includes acts of containing nucleic acid in a plurality of microfluidic droplets, and evenly amplifying at least some of the nucleic acid contained within the microfluidic droplets.

In one set of embodiments, the method comprises containing a plurality of nucleic acids in a first plurality of microfluidic droplets, amplifying at least some of the nucleic acids within the first plurality of microfluidic droplets, combining the amplified nucleic acids in a common solution, containing the amplified nucleic acids in a second plurality of microfluidic droplets, and amplifying at least some of the amplified nucleic acids within the second plurality of microfluidic droplets.

The method, in one embodiment, is generally directed to evenly amplifying a plurality of nucleic acids contained within microfluidic droplets.

In yet another set of embodiments, the method comprises mixing first microfluidic droplets containing a species of interest with second microfluidic droplets free of the species of interest to produce a mixture of microfluidic droplets, and transferring (for example, pipetting) at least 10 nl of the mixture of microfluidic droplets into a container.

In some embodiments, the method includes acts of mixing first microfluidic droplets containing a species of interest with second microfluidic droplets free of the species of interest to produce a mixed fluid containing the microfluidic droplets to produce a mixture of microfluidic droplets, and transferring, on average, a plurality of second microfluidic droplets and no more than about 1.5 first microfluidic droplets into a container.

In another aspect, the present invention is generally directed to a kit. In one set of embodiments, the kit includes a droplet-making device configured to produce microfluidic droplets, a microfluidic device configured to manipulate the microfluidic droplets, and a container containing a plurality of microfluidic droplets having substantially the same composition.

In another aspect, the present invention encompasses methods of making one or more of the embodiments described herein. In still another aspect, the present invention encompasses methods of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrate amplification of nucleic acids contained within droplets, in certain embodiments of the invention;

FIG. 2 illustrate transferal of a droplet of interest, in another embodiment of the invention;

FIG. 3 illustrates an apparatus for use with certain embodiments of the invention;

FIG. 4 illustrates a schematic for PCR amplification within droplets, according to some embodiments of the invention;

FIG. 5 illustrates evenly amplified nucleic acids, in another embodiment of the invention;

FIG. 6 illustrates a schematic for PCR amplification within droplets, according to some embodiments of the invention;

FIGS. 7A-7B illustrate amplified nucleic acids, in yet another embodiment of the invention; and

FIG. 8 illustrates amplification in droplets in accordance with another embodiment of the invention.

### DETAILED DESCRIPTION

The present invention generally relates to droplet-based microfluidic devices, including systems, methods, and kits for amplifying or cloning within droplets. In some embodiments, the present invention is generally directed to systems, methods, or kits for amplifying a plurality of nucleic acids, e.g., without substantially selectively amplifying some nucleic acids over others. The nucleic acids may be contained within the droplets. In addition, in some embodiments, a plurality of microfluidic droplet containing a species of interest, such as a nucleic acid, may be mixed with microfluidic droplets free of the species, then pipetted or otherwise transferred such that, on average, a predetermined number of droplets containing species of interest is transferred.

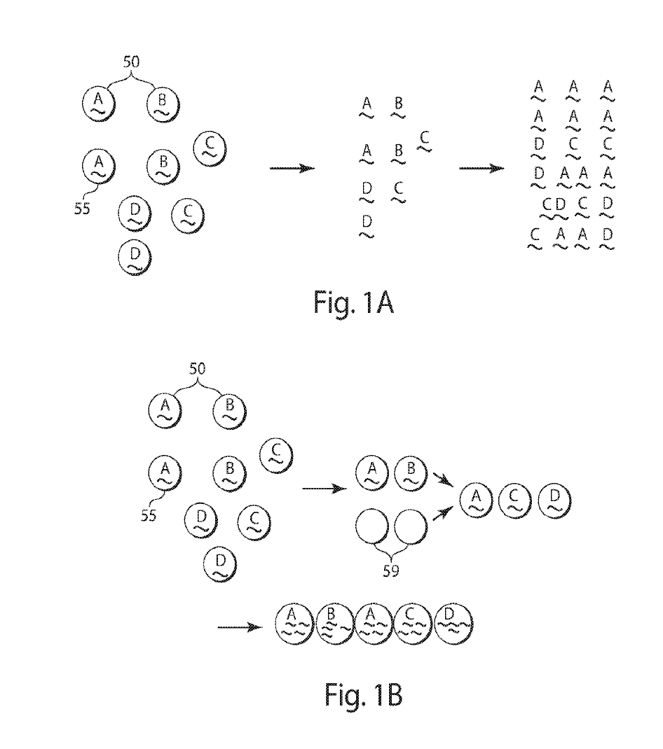

Referring now to FIG. 1, one aspect of the present invention for amplifying nucleic acids contained within droplets is shown. In this figure, a plurality of cells 50 containing nucleic acids to be amplified 55 is shown. The cells may be the same or different, and the cells may have the same or different nucleic acids. For example, one (or more) of the cells may be a cancerous cell with a mutated genome, e.g., within a population of normal cells. However, it should be understood that this is by way of example only; in some embodiments, free DNA or other nucleic acids may be used, e.g., without necessarily arising from a predetermined cell. For instance, the nucleic acids may arise from forensic DNA sample analysis or other unknown sources or unknown cells.

If cells 50 were to be lysed and their nucleic acids collected together for amplification, then the nucleic acids from the different cells would also be mixed together. In some cases, amplification of such a mixture of different nucleic acids can create problems during amplification. For example, as is shown in FIG. 1A, certain nucleic acids may be selectively amplified over others due to competition effects, resulting in a variety of errors, such as a skewed distribution (e.g., strand A), loss of species (e.g., strand B), or “chimeras” (e.g., strands C/D) created during the amplification process. For example, chimeras may be created by cross-hybridization of two templates or by the dissociation of an enzyme from a nucleic acid template onto a different nucleic acid template during the growth process. In addition, some strands may be selectively amplified over other strands, e.g., due to differences in enzymatic affinity, random processes and variability during amplification, or the like. Thus, the amplified nucleic acids may have low fidelity as compared to the original nucleic acid population.

However, in some embodiments of the invention, these problems may be avoided or reduced through the amplification of nucleic acids contained within droplets. For example, as is shown in FIG. 1B, a population of droplets containing nucleic acids may be exposed to conditions suitable to cause amplification of the nucleic acids within the droplets. For instance, the droplets may be merged with droplets 59 containing suitable compounds for amplification purposes, such as polymerases and/or deoxyribonucleotides, and/or by exposing the droplets to suitable temperature changes. Techniques for merging droplets are known to those of ordinary skill in the art. As a non-limiting example, two or more fluids, carrying different reagents and/or templates can be introduced into droplets simultaneously using co-flow microfluidic devices; for instance, one fluid carries PCR reagents and second fluid carries template mix. As each amplification reaction for each nucleic acid occurs separately within each droplet, without mixing of different nucleic acids together, fidelity may be substantially maintained. Thus, errors such as skewed distribution, loss of sample, or chimeras may be substantially reduced or eliminated, as amplification of each nucleic acid generally stays within each droplet, without mixing of other nucleic acids.

Accordingly, certain aspects of the present invention are generally directed to systems and methods for amplifying nucleic acids contained within droplets, e.g., for sequencing or other applications. The nucleic acids may be, for example, RNA and/or DNA, such as genomic DNA or mitochondrial DNA. In some cases, the nucleic acids are free-floating or contained within a fluid contained within the droplet. The nucleic acid may be taken from one or more cells (e.g., released upon lysis of one or more cells), synthetically produced, or the like. If the nucleic acid arises from cells, the cells may come from the same or different species (e.g., mouse, human, bacterial, etc.), and/or the same or different individual. For example, the nucleic acids may come from cells of a single organism, e.g., healthy or diseased cells (e.g., cancer cells), different organs of the organism, etc. In some cases, different organisms may be used (e.g., of the same or different species). In some cases, the nucleic acids may have a distribution such that some nucleic acids are not commonly present within a nucleic acid population. For example, there may be one cancer or disease cell among tens, hundreds, thousands, or more of normal or other cells.

The nucleic acids may be contained within droplets, and may be amplified within the droplets. In some embodiments, the nucleic acids may first be fragmented prior to encapsulation within droplets. For instance, the nucleic acids may be released from cells, e.g., upon cell lysis, then fragmented using techniques such as ultrasound or mechanical disruption. The cells, if used, may be contained within droplets prior to lysis, or the cells may be first lysed then the cell lysates contained within one or more droplets. Techniques for encapsulating nucleic acids (or cells) within droplets are known to those of ordinary skill in the art.

A variety of techniques can be used to amplify the nucleic acids within droplets, such as PCR (polymerase chain reaction) techniques. However, by amplifying the nucleic acids within the droplets, e.g., prior to releasing the nucleic acids from the droplets, “even” amplification of the various nucleic acids may be achieved in some embodiments of the invention. Generally, in “even” amplification, approximately the same amount of nucleic acids may be produced within each droplet. In contrast, if a variety of nucleic acids are mixed together in bulk and then amplified (e.g., as is typically performed in PCR), differences in reaction rate between the various nucleic acids during PCR may result in some nucleic acids being amplified over other nucleic acids, and in some cases, some of the nucleic acids may be lost due to relative overamplification by the other nucleic acids. See, e.g., FIG. 1A. Thus, for instance, nucleic acids that may react more slowly (e.g., upon exposure to a polymerase or other enzymes) may be amplified under “even” amplification conditions, in contrast to bulk amplification.

Thus, according to certain embodiments of the present invention, the nucleic acids within a plurality of droplets may be amplified “evenly,” such that the distribution of nucleic acids is not substantially changed after amplification, relative to before amplification. The droplets may be fluidic droplets, e.g., as discussed herein. For example, according to certain embodiments, the nucleic acids within a plurality of droplets may be amplified such that the number of nucleic acid molecules for each type of nucleic acid may have a distribution such that, after amplification, no more than about 5%, no more than about 2%, or no more than about 1% of the nucleic acids have a number less than about 90% (or less than about 95%, or less than about 99%) and/or greater than about 110% (or greater than about 105%, or greater than about 101%) of the overall average number of amplified nucleic acid molecules per droplet. In some embodiments, the nucleic acids within the droplets may be amplified such that each of the nucleic acids that are amplified can be detected in the amplified nucleic acids, and in some cases, such that the mass ratio of the nucleic acid to the overall nucleic acid population changes by less than about 50%, less than about 25%, less than about 20%, less than about 15%, less than about 10%, or less than about 5% after amplification, relative to the mass ratio before amplification. In some cases, amplification fidelity may be determined by breaking the droplets, releasing the nucleic acids, and performing hybridization on the nucleic acids, or a FISH test may be performed on the nucleic acids.

As mentioned, PCR (polymerase chain reaction) or other amplification techniques may be used to amplify nucleic acids, e.g., contained within droplets. Typically, in PCR reactions, the nucleic acids are heated (e.g., to a temperature of at least about 50° C., at least about 70° C., or least about 90° C. in some cases) to cause dissociation of the nucleic acids into single strands, and a heat-stable DNA polymerase (such as Taq polymerase) is used to amplify the nucleic acid. This process is often repeated multiple times to amplify the nucleic acids. Those of ordinary skill in the art will be aware of a variety of different PCR techniques described in the scientific literature.

Thus, in one set of embodiments, PCR amplification may be performed within the droplets. For example, the droplets may contain a polymerase (such as Taq polymerase), and DNA nucleotides (deoxyribonucleotides), and the droplets may be processed (e.g., via repeated heated and cooling) to amplify the nucleic acid within the droplets. Suitable reagents for PCR or other amplification techniques, such as polymerases and/or deoxyribonucleotides, may be added to the droplets during their formation, and/or afterwards (e.g., via merger with droplets containing such reagents, and/or via direct injection of such reagents, e.g., contained within a fluid). Various techniques for droplet injection or merger of droplets will be known to those of ordinary skill in the art. See, e.g., U.S. Pat. Apl. Pub. No. 2012/0132288, incorporated herein by reference. In addition, in some cases, suitable primers may be used to initiate polymerization, e.g., P5 and P7, or other primers known to those of ordinary skill in the art. In some embodiments, primers may be added to the droplets, or the primers may be present on one or more of the nucleic acids within the droplets. Those of ordinary skill in the art will be aware of suitable primers, many of which can be readily obtained commercially.

For instance, as a non-limiting example, a droplet may contain polymerase and DNA nucleotides, which is fused to a droplet containing nucleic acids, to allow amplification of the nucleic acids to occur. Those of ordinary skill in the art will be aware of suitable PCR techniques and variations, such as assembly PCR or polymerase cycling assembly, which may be used in some embodiments to produce an amplified nucleic acid.

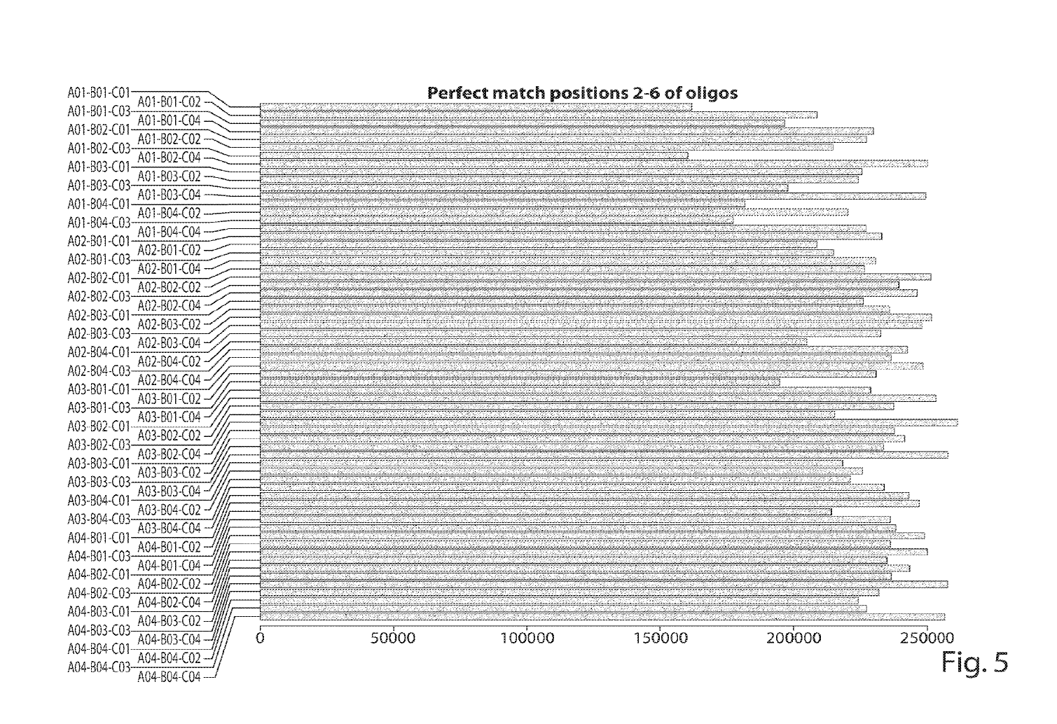

The nucleic acids may be amplified to any suitable extent. The degree of amplification may be controlled, for example, by controlling factors such as the temperature, cycle time, or amount of enzyme and/or deoxyribonucleotides contained within the droplets. For instance, in some embodiments, a population of droplets may have at least about 50,000, at least about 100,000, at least about 150,000, at least about 200,000, at least about 250,000, at least about 300,000, at least about 400,000, at least about 500,000, at least about 750,000, at least about 1,000,000 or more molecules of the amplified nucleic acid per droplet. See, e.g., FIG. 5 for an example of a population of nucleic acid molecules that have been evenly amplified within droplets.

In some cases, the droplets may be burst or disrupted, e.g., to sequence the nucleic acids contained within the droplets. For example, droplets contained in a carrying fluid may be disrupted using techniques such as mechanical disruption, chemical disruption, and/or ultrasound. Examples of methods for sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing, nanopore sequencing, microfluidic Sanger sequencing, digital RNA sequencing (“digital RNA-seq”), etc.

As mentioned, certain aspects of the present invention involve the use of a plurality of droplets containing cells, and/or nucleic acids (such as genomic DNA) arising from cells. The cells may be substantially identical or different. For example, a droplet may contain more than one cell or other species, where the cells (or other species) are the same or different; the cells (or other species) in different droplets may also be the same or different. If cells are used, the cells may also be, in some embodiments, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like. The droplets may be fluidic droplets, e.g., as discussed herein.

In some embodiments, one or more “tags” may be present within a droplet, which can be analyzed or used, for instance, to determine the identity and/or history of the droplet, to determine cells or other species in the droplets, to determine nucleic acids within the droplet, or the like. In some cases, the tags may be chosen to be relatively inert relative to other components of the droplet. The tags may be present initially in the droplet, and/or subsequently added. For instance, tags may be added when the droplet is exposed to one or more conditions (or proximate in time to such exposure). In some cases, more than one tag may be present in a droplet. Non-limiting examples of suitable conditions include those discussed in U.S. Pat. Apl. Ser. No. 61/981,123, entitled “Systems and Methods for Droplet Tagging,” by Bernstein, et al., filed Apr. 17, 2014; U.S. Pat. Apl. Ser. No. 61/981,108, entitled “Methods and Systems for Droplet Tagging and Amplification,” by Weitz, et al., filed Apr. 17, 2014, each incorporated herein by reference in its entirety.

In certain embodiments of the invention, the tags within a droplet can be joined together, for example, chemically, to produce a joined tag. The tags may be free-floating within a fluid contained within the droplet. Any suitable technique may be used to join tags together, e.g., prior to removal from the droplet. The tags may be joined using any suitable technique. For example, the tags may be joined together using an enzyme, a catalyst, or a reactant, which may be added to the droplet using any suitable technique. For instance, a droplet containing the tags may be fused to another droplet containing the chemical agent, or a chemical reactant may be added or inserted into a droplet, for example, using pipetting or other techniques, and in some cases, using automated techniques.

By joining the tags in a droplet together to produce a joined tag, the identity and/or history of the droplet may be maintained by maintaining the joined tags, even if the tags are separated from the droplet or tags from different droplets are mixed together. For example, joined tags from a variety of droplets can be collected together and analyzed. In some embodiments, a series of droplets may be separated into various groups depending on various properties, and the tags within each group may be manipulated together and/or used to identify such droplets having such properties.

The tags may include, for example, nucleic acids, which may be joined together. In one set of embodiments, the nucleic acids may be joined together using enzymes. For instance, in certain embodiments, the nucleic acids together are joined together using ligases. Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, Taq DNA Ligase, or the like. Many such ligases may be purchased commercially. As additional examples, in some embodiments, two or more nucleic acids may be ligated together using annealing or a primer extension method. In yet another set of embodiments, the nucleic acids may be joined together and or amplified using PCR (polymerase chain reaction) or other amplification techniques.

In some embodiments, various sequences of nucleic acids acting as tags can be used to encode specific identities and/or conditions that a droplet may be exposed to (for example, nucleic acids arising from a cell, or other nucleic acids as discussed herein), and such nucleic acid tags can be added thereto to indicate such exposure to a condition, in accordance with certain embodiments. In some cases, the nucleic acids within a droplet may be joined together prior to removal (for example, upon bursting of a droplet, washing of a slide, etc.). Different nucleic acids from different droplets may be mixed together; however, even after such mixing, each nucleic acid can be individually sequenced to determine the specific conditions that the corresponding droplet had been exposed to.

Any suitable system may be used for encoding. For example, in one set of embodiments, a nucleic acid tag may include an encoding region of nucleotides, and optionally a connecting region. The nucleotides in the encoding region may correspond to a specific condition (or set of conditions). Any suitable number of conditions may be arbitrarily encoded in such a fashion, where the number of conditions that are encodable by such an encoding region may be determined by the number of nucleotides in the encoding region. Thus, for instance, an encoding region having length n can encode up to 4ⁿ regions (based on the four types of nucleotides). For example, a first condition may be encoded with A, a second condition may be encoded with T (or U if the nucleic acid is an RNA), a third condition may be encoded with G, a fourth condition may be encoded with C, etc. As a more complex example, an encoding region containing 3 nucleotides is sufficient to encode over 50 different conditions (since 4³=64). One or more than one encoding region may be used. In addition, the encoding region may also include other nucleotides used for error detection and/or correction, redundancy, or the like, in certain embodiments.

A nucleic acid tag may also include, in some cases, one or more connecting regions, which are joined together. For example, the connecting regions may include “sticky ends,” or overhangs of nucleic acids, such that only specific nucleic acids can be properly joined together. For example a first nucleic acid tag (encoding a first condition) may include a first sticky end that is substantially complementary to a sticky end on a second nucleic acid tag but not to a sticky end on the third nucleic acid tag; similarly, a second nucleic acid (encoding a second condition) may include a sticky end that is substantially complementary to a sticky end on a third nucleic acid tag (encoding a third condition) but not to the sticky end on the first nucleic acid. Thus, upon exposure to suitable ligases, the first, second, and third nucleic acids may be joined together in an order suitable for subsequent study, without the nucleic acids being incorrectly joined together in an incorrect order (e.g., a first nucleic acid being joined to another first nucleic acid). Accordingly, by sequencing the final joined nucleic acid, it can be determined that this nucleic acid was in a droplet exposed to the first, second, and third conditions. However, it should be understood that in other embodiments, there may be no need to ensure that the nucleic acid tags are joined together in a certain configuration or order.

The nucleic acid tag may also have any suitable length or number of nucleotides, depending on the application. For example, a nucleic acid tag may have a length shorter or longer than 10 nt, 30 nt, 50 nt, 100 nt, 300 nt, 500 nt, 1000 nt, 3000 nt, 5000 nt, or 10000 nt, etc. In some cases, other portions of the nucleic acid tag may also be used for other purposes, e.g., in addition to encoding conditions. For example, portions of the nucleic acid tag may be used to increase the bulk of the nucleic acid tag (e.g., using specific sequences or nonsense sequences), to facilitate handling (for example, a tag may include a poly-A tail), to increase selectivity of binding (e.g., as discussed below), to facilitate recognition by an enzyme (e.g., a suitable ligase), to facilitate identification, or the like.

In some cases, the droplets may be burst or disrupted, e.g., to sequence the nucleic acids contained within the droplets. For example, droplets contained in a carrying fluid may be disrupted using techniques such as mechanical disruption, chemical disruption, and/or ultrasound. If tags are present, the tags may then be determined to determine the identity and/or history of the droplet, e.g., to determine conditions that the droplet was exposed to. Any suitable method can be used to determine the tags, depending on the type of tags used. For example, fluorescent particles may be determined using fluorescence measurements, or nucleic acids may be sequenced using a variety of techniques and instruments, many of which are readily available commercially. Non-limiting examples of techniques for sequencing nucleic acids include those described herein.

In some embodiments, multiple rounds of encapsulation into droplets and droplet disruption may occur. For example, in some cases, nucleic acids (such as those discussed herein) may be encapsulated in a first plurality of droplets, then the droplets later disrupted and their interiors pooled together, e.g., in a common solution. The interiors may then be encapsulated within a second plurality of droplets.

In some embodiments, nucleic acids from any suitable source may be contained within a first plurality of microfluidic droplets, and amplified or manipulated in some way within the droplets, e.g., as discussed herein. In some cases, the amplified nucleic acids (or “amplicons”) may be combined together, e.g., by bursting or disrupting the droplets into a common solution, and the solution may then be contained within a second plurality of microfluidic droplets.

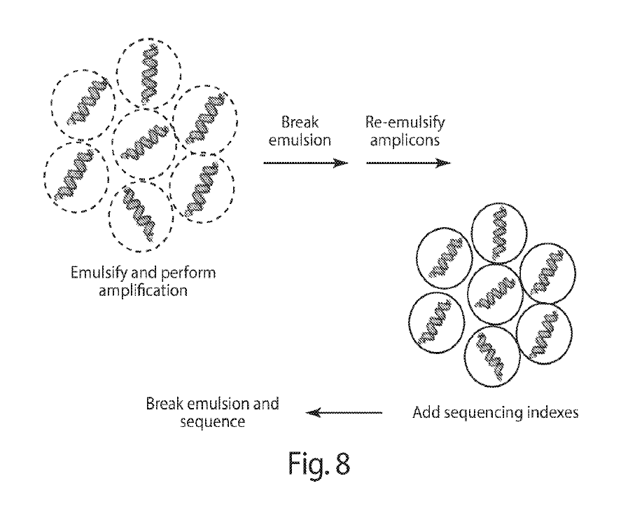

One non-limiting example is illustrated with reference to FIG. 8. In FIG. 8, a plurality of nucleic acids (e.g., arising from a fragmented nucleic acid, biological templates, or other suitable sources) is encapsulated within a first plurality of droplets. The nucleic acids may include, e.g., DNA or RNA. Techniques for encapsulating nucleic acids in droplets include any of those discussed herein.

In some cases, the nucleic acids may be manipulated in some fashion within the droplets. Examples include any of those discussed herein. For instance, in one set of embodiments, various chemicals or other species may be added to the droplets, for example, primers, nucleotides, other nucleic acids, dyes, or the like. As a non-limiting example, the nucleic acids within the droplets may be exposed to conditions to allow amplification of the nucleic acids (e.g., to produce “amplicons”) within the droplets to occur (e.g., such that even amplification occurs, as discussed herein). In some cases, sorting or merging of the microfluidic droplets may occur.

As shown in FIG. 8, after amplification, the droplets may be broken or otherwise disrupted, e.g., as discussed herein, and the nucleic acid within the droplets combined, e.g., within a common solution. In some embodiments, the nucleic acids may be manipulated in some fashion in solution, or various chemicals or other species may be added or removed. In some cases, portions or aliquots of the solution may also be removed, e.g., for subsequent assays. For example, in one embodiment, the nucleic acids may be purified in solution and other species (e.g., unreacted species, catalysts or enzymes, etc.) may be removed.

The nucleic acids may then be encapsulated within a second plurality of droplets, e.g., as shown in FIG. 8. Similar to the above, the nucleic acids may be manipulated in some fashion within the droplets, e.g., as discussed herein. For instance, in one set of embodiments, various chemicals or other species may be added to the droplets, for example, primers, nucleotides, other nucleic acids, dyes, or the like. As a non-limiting example, the nucleic acids within the droplets may be exposed to conditions to allow amplification of the nucleic acids within the droplets to occur (e.g., for indexing or sequencing). For instance, in some embodiments, “barcodes” such as those described herein may be added to the droplets, e.g., to tag the nucleic acids within the droplets. In some cases, sequencing may occur within the droplets, although in some cases, the droplets may then be broken or otherwise disrupted prior to sequencing the nucleic acids. In some cases, sorting or merging of the microfluidic droplets may also occur.

Additionally, it should be understood that the above is not meant to be limiting. For example, in some embodiments, amplification and incorporation with barcodes may both be performed within a plurality of microfluidic droplets, i.e., without necessarily requiring bursting of the droplets between these.

Some aspects of the present invention are generally directed to systems and methods for transferring a microfluidic droplet to a container, e.g., for further analysis or study. The transferring may include, for example, pipetting, and the transferring may be performed, for example, manually or automatically. The microfluidic droplet may contain a species of interest, such as a nucleic acid (such as those described herein) or a cell or other sample, and the container may be, for example, a vial, a test tube, a beaker, a well of a microwell plate (e.g., a 96-well plate, a 384-well plate, a 1,536-well plate, etc.), or the like.

In some embodiments, the container that the droplet is to be transferred to is of macroscopic dimensions. For example, the container, may be used to analyze a sample using ordinary (macroscale) laboratory equipment (e.g., plate readers, spectrofluorimeters, balances, centrifuges, etc.). However, the microfluidic droplet may often be of a very small size (e.g., having an average diameter of less than about 1 mm or a volume of less than about 1 microliter). Accordingly, there are significant challenges in pipetting or otherwise transferring such a microfluidic droplet into such containers, for example, the difficulty in accurately pipetting or otherwise transferring small volumes, or the difficulty in separating or isolating the microfluidic droplet from other microfluidic droplets, e.g., within a fluid.

The microfluidic droplet to be transferred may be of any suitable diameter or volume. For instance, the microfluidic droplet, in some cases, may be less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 5 micrometers, less than about 3 micrometers, less than about 1 micrometer, etc. The average dimension may also be greater than about 1 micrometer, greater than about 3 micrometers, greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in some cases. Combinations of any of these are also possible; for example, the average or characteristic dimension of the microfluidic droplet may be between about 1 mm and about 100 micrometers.

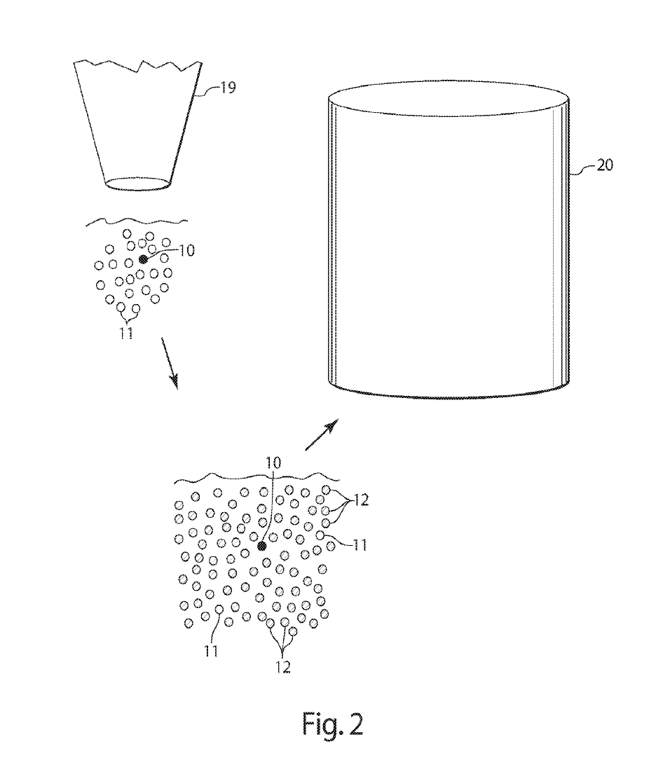

In one set of embodiments, the microfluidic droplet of interest may be mixed with other, second microfluidic droplets that are not of interest. For instance, as is shown in FIG. 2, a droplet of interest 10 contained in fluid 15 (e.g., a liquid) is to be transferred to a container 20 (for example, a vial or a well of a microwell plate). The droplet of interest may be a microfluidic droplet, of relatively small dimensions, and in some cases, the droplet may be surrounded by other droplets 11. However, only the droplet of interest 10 is desired to be transferred, e.g., without also simultaneously transferring the other droplets into the same destination container.

Pipettes and other macroscale laboratory equipment (e.g., syringes, eyedroppers, etc.) cannot ordinary be used in such a fashion. For instance, as is depicted in FIG. 2, the amount of fluid taken up by a pipette 19 is usually significantly larger than the microfluidic droplet, and it is not possible to withdrawal only droplet of interest 10 from fluid 15 without also accidentally withdrawing one or more of droplets 11 simultaneously. (However, note that FIG. 2 is not drawn to scale.) However, in certain embodiments, a plurality of second microfluidic droplets 12 may be added to fluid 15. In some embodiments, the volume of fluid 15 may be increased (i.e., with or without adding droplets 12). This may have the effect of “diluting” the droplet of interest 10 from the other droplets 11, as is shown in FIG. 2 with droplets 12. In some cases, the second microfluidic droplets may be substantially free of the species of interest, and/or free of species similar to the species of the interest. For example, if the microfluidic droplet of interest contains a specific nucleic acid (or a nucleic acid fragment, e.g., from a genome), then the second microfluidic droplets may be free of the specific nucleic acid and/or free of other nucleic acids. Thus, upon transfer, on the average, only one (or a small or predetermined number) of droplets of interest 10 are transferred to container 20, without also transferring (or transferring a smaller number of) other droplets 11.

The composition of the second microfluidic droplets may be the same or different from the microfluidic droplet of interest. Similarly, the second microfluidic droplets may independently have the same or different compositions from each other. The second microfluidic droplets may be substantially monodisperse, and/or have a range of sizes or average diameters, which may be the same or different from the diameter of the microfluidic droplet of interest.

For instance, in one set of embodiments, the second microfluidic droplets may have a distribution of average diameters such that no more than about 20%, no more than about 10%, or no more than about 5% of the droplets may have an average diameter greater than about 120% or less than about 80%, greater than about 115% or less than about 85%, greater than about 110% or less than about 90%, greater than about 105% or less than about 95%, greater than about 103% or less than about 97%, or greater than about 101% or less than about 99% of the average diameter of the second microfluidic droplets. The “characteristic dimension” of a droplet, as used herein, is the diameter of a perfect sphere having the same volume as the droplet. In addition, in some instances, the coefficient of variation of the average diameter of the droplets may be less than or equal to about 20%, less than or equal to about 15%, less than or equal to about 10%, less than or equal to about 5%, less than or equal to about 3%, or less than or equal to about 1%. However, as previously discussed, in other embodiments, the second microfluidic droplets may not necessarily be substantially disperse, and may instead exhibit a range of different diameters.

The average diameter of the second microfluidic droplets, in some embodiments, may be less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 5 micrometers, less than about 3 micrometers, less than about 1 micrometer, etc. The average diameter may also be greater than about 1 micrometer, greater than about 3 micrometers, greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in certain cases. Combinations of any of these are also possible. Thus, for example, the average or characteristic dimension of the second microfluidic droplets may be between about 1 mm and about 10 micrometers.

In some cases, the microfluidic droplet of interest may be mixed with the second microfluidic droplets of interest at a ratio of at least about 1:10, at least about 1:30, at least about 1:50, at least about 1:100, at least about 1:300, at least about 1:500, at least about 1:1,000, at least about 1:3,000, at least about 1:500, at least about 1:10,000, at least about 1:30,000, at least about 1:50,000, at least about 1:100,000, at least about 1:300,000, at least about 1:500,000, at least about 1:000,000, or any other suitable ratio. In some cases, relatively high ratios are used, e.g., such that the microfluidic droplet of interest is substantially separated from other microfluidic droplets that may be of interest.

Accordingly, as is shown in FIG. 2, the droplet of interest 10 is now widely spaced or diluted, relative to other droplets 11, by additional fluid and/or second microfluidic droplets 12. It should be noted that although additional fluid could have been added to fluid 15 (i.e., without adding the second microfluidic droplets), doing so could in some cases also alter the physical properties or characteristics of fluid 15, in some instances relatively adversely. However, in other cases, fluid (without droplets 12) may also be used to “dilute” droplet 10 from other droplets 11.

In some cases, when a quantity of fluid 15 is transferred to container 20 (e.g., using pipette 19), on the average, only a single droplet of interest 10 is transferred (along with second microfluidic droplets 12), but without any of droplets 11. However, it should be noted that while only a single droplet of interest 10 was transferred in this example, other suitable numbers of droplets of interest may also be transferred in other embodiments. For example, in some cases, the droplet of interest may be mixed with second microfluidic droplets such that, on the average, no more than about 10 of the droplets of interest are transferred, e.g., into a container. As mentioned, this is typically determined “on average”; a single transfer may contain more or fewer microfluidic droplets of interest, e.g., due to random probability or sampling, mixing within the fluid, etc. In some cases, for example, less than about 1,000,000 droplets of interest, less than about 500,000 droplets of interest less than about 300,000 droplets of interest, less than about 100,000 droplets of interest, less than about 50,000 droplets of interest, less than about 30,000 droplets of interest, less than about 10,000 droplets of interest, less than about 5,000 droplets of interest, less than about 3,000 droplets of interest, less than about 1,000 droplets of interest, less than about 500 droplets of interest, less than about 300 droplets of interest, less than about 100 droplets of interest, less than about 50 droplets of interest, less than about 30 droplets of interest, less than about 10 droplets of interest, less than about 5 droplets of interest, less than about 3 droplets of interest, less than about 2 droplets of interest, less than about 1.5 droplets of interest, less than about 1 droplet of interest, less than about 0.5 droplets of interest, less than about 0.3 droplets of interest, less than about 0.1 droplets of interest, etc. may be transferred, e.g., into a suitable container. (Fractions of droplets are also possible, as this is determined “on average.”)

In addition, the volume of liquid transferred may depend on the application; for example, in some cases, the volume transferred may be at least about 10 nl, at least about 30 nl, at least about 50 nl, at least about 100 nl, at least about 300 nl, at least about 500 nl, at least about 1 microliter, at least about 3 microliters, at least about 5 microliters, at least about 10 microliters, at least about 30 microliters, at least about 50 microliters, at least about 100 microliters, at least about 300 microliters, at least about 500 microliters, at least about 1 ml, etc. In some cases, the volume transferred may be no more than about 1 ml, no more than about 500 microliters, no more than about 300 microliters, no more than about 100 microliters, no more than about 50 microliters, no more than about 30 microliters, no more than about 10 microliters, no more than about 5 microliters, no more than about 3 microliters, no more than about 1 microliters, no more than about 500 nl, no more than about 300 nl, no more than about 100 nl, no more than about 50 nl, no more than about 30 nl, no more than about 10 nl, etc. may be transferred. Combinations of any of these are also possible, e.g., the volume transferred may be between 300 microliters and 500 microliters of fluid. In some cases, the volume of microfluidic droplets that are transferred may be any of the values or ranges given above.

Additional details regarding systems and methods for manipulating droplets in a microfluidic system follow, e.g., for determining droplets (or species within droplets), sorting droplets, merging or coalescing droplets, etc. The droplets may be microfluidic droplets, e.g., containing a fluid, and may be surrounded by a second fluid, e.g., substantially immiscible with the fluid contained within the droplet. The fluid may be a liquid, e.g., an aqueous liquid. In some embodiments, the droplet is not a gel or in a semi-solid state. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some aspects, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

In certain embodiments of the invention, sensors are provided that can sense and/or determine one or more characteristics of the fluidic droplets, and/or a characteristic of a portion of the fluidic system containing the fluidic droplet (e.g., the liquid surrounding the fluidic droplet) in such a manner as to allow the determination of one or more characteristics of the fluidic droplets. Characteristics determinable with respect to the droplet and usable in the invention can be identified by those of ordinary skill in the art. Non-limiting examples of such characteristics include fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), or the like.

In some cases, the sensor may be connected to a processor, which in turn, cause an operation to be performed on the fluidic droplet, for example, by sorting the droplet, adding or removing electric charge from the droplet, fusing the droplet with another droplet, splitting the droplet, causing mixing to occur within the droplet, etc., for example, as previously described. For instance, in response to a sensor measurement of a fluidic droplet, a processor may cause the fluidic droplet to be split, merged with a second fluidic droplet, etc.

One or more sensors and/or processors may be positioned to be in sensing communication with the fluidic droplet. “Sensing communication,” as used herein, means that the sensor may be positioned anywhere such that the fluidic droplet within the fluidic system (e.g., within a channel), and/or a portion of the fluidic system containing the fluidic droplet may be sensed and/or determined in some fashion. For example, the sensor may be in sensing communication with the fluidic droplet and/or the portion of the fluidic system containing the fluidic droplet fluidly, optically or visually, thermally, pneumatically, electronically, or the like. The sensor can be positioned proximate the fluidic system, for example, embedded within or integrally connected to a wall of a channel, or positioned separately from the fluidic system but with physical, electrical, and/or optical communication with the fluidic system so as to be able to sense and/or determine the fluidic droplet and/or a portion of the fluidic system containing the fluidic droplet (e.g., a channel or a microchannel, a liquid containing the fluidic droplet, etc.). For example, a sensor may be free of any physical connection with a channel containing a droplet, but may be positioned so as to detect electromagnetic radiation arising from the droplet or the fluidic system, such as infrared, ultraviolet, or visible light. The electromagnetic radiation may be produced by the droplet, and/or may arise from other portions of the fluidic system (or externally of the fluidic system) and interact with the fluidic droplet and/or the portion of the fluidic system containing the fluidic droplet in such as a manner as to indicate one or more characteristics of the fluidic droplet, for example, through absorption, reflection, diffraction, refraction, fluorescence, phosphorescence, changes in polarity, phase changes, changes with respect to time, etc. As an example, a laser may be directed towards the fluidic droplet and/or the liquid surrounding the fluidic droplet, and the fluorescence of the fluidic droplet and/or the surrounding liquid may be determined. “Sensing communication,” as used herein may also be direct or indirect. As an example, light from the fluidic droplet may be directed to a sensor, or directed first through a fiber optic system, a waveguide, etc., before being directed to a sensor.

Non-limiting examples of sensors useful in the invention include optical or electromagnetically-based systems. For example, the sensor may be a fluorescence sensor (e.g., stimulated by a laser), a microscopy system (which may include a camera or other recording device), or the like. As another example, the sensor may be an electronic sensor, e.g., a sensor able to determine an electric field or other electrical characteristic. For example, the sensor may detect capacitance, inductance, etc., of a fluidic droplet and/or the portion of the fluidic system containing the fluidic droplet.

As used herein, a “processor” or a “microprocessor” is any component or device able to receive a signal from one or more sensors, store the signal, and/or direct one or more responses (e.g., as described above), for example, by using a mathematical formula or an electronic or computational circuit. The signal may be any suitable signal indicative of the environmental factor determined by the sensor, for example a pneumatic signal, an electronic signal, an optical signal, a mechanical signal, etc.

In one set of embodiments, a fluidic droplet may be directed by creating an electric charge and/or an electric dipole on the droplet, and steering the droplet using an applied electric field, which may be an AC field, a DC field, etc. As an example, an electric field may be selectively applied and removed (or a different electric field may be applied, e.g., a reversed electric field) as needed to direct the fluidic droplet to a particular region. The electric field may be selectively applied and removed as needed, in some embodiments, without substantially altering the flow of the liquid containing the fluidic droplet. For example, a liquid may flow on a substantially steady-state basis (i.e., the average flowrate of the liquid containing the fluidic droplet deviates by less than 20% or less than 15% of the steady-state flow or the expected value of the flow of liquid with respect to time, and in some cases, the average flowrate may deviate less than 10% or less than 5%) or other predetermined basis through a fluidic system of the invention (e.g., through a channel or a microchannel), and fluidic droplets contained within the liquid may be directed to various regions, e.g., using an electric field, without substantially altering the flow of the liquid through the fluidic system.

In some embodiments, the fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc.

In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. The liquid reservoirs may be positioned such that, when activated, the movement of liquid caused by the activated reservoirs causes the liquid to flow in a preferred direction, carrying the fluidic droplet in that preferred direction. For instance, the expansion of a liquid reservoir may cause a flow of liquid towards the reservoir, while the contraction of a liquid reservoir may cause a flow of liquid away from the reservoir. In some cases, the expansion and/or contraction of the liquid reservoir may be combined with other flow-controlling devices and methods, e.g., as described herein. Non-limiting examples of devices able to cause the expansion and/or contraction of a liquid reservoir include pistons and piezoelectric components. In some cases, piezoelectric components may be particularly useful due to their relatively rapid response times, e.g., in response to an electrical signal. In some embodiments, the fluidic droplets may be sorted into more than two channels.

As mentioned, certain embodiments are generally directed to systems and methods for sorting fluidic droplets in a liquid, and in some cases, at relatively high rates. For example, a property of a droplet may be sensed and/or determined in some fashion (e.g., as further described herein), then the droplet may be directed towards a particular region of the device, such as a microfluidic channel, for example, for sorting purposes. In some cases, high sorting speeds may be achievable using certain systems and methods of the invention. For instance, at least about 10 droplets per second may be determined and/or sorted in some cases, and in other cases, at least about 20 droplets per second, at least about 30 droplets per second, at least about 100 droplets per second, at least about 200 droplets per second, at least about 300 droplets per second, at least about 500 droplets per second, at least about 750 droplets per second, at least about 1,000 droplets per second, at least about 1,500 droplets per second, at least about 2,000 droplets per second, at least about 3,000 droplets per second, at least about 5,000 droplets per second, at least about 7,500 droplets per second, at least about 10,000 droplets per second, at least about 15,000 droplets per second, at least about 20,000 droplets per second, at least about 30,000 droplets per second, at least about 50,000 droplets per second, at least about 75,000 droplets per second, at least about 100,000 droplets per second, at least about 150,000 droplets per second, at least about 200,000 droplets per second, at least about 300,000 droplets per second, at least about 500,000 droplets per second, at least about 750,000 droplets per second, at least about 1,000,000 droplets per second, at least about 1,500,000 droplets per second, at least about 2,000,000 or more droplets per second, or at least about 3,000,000 or more droplets per second may be determined and/or sorted.

In some aspects, a population of relatively small droplets may be used. In certain embodiments, as non-limiting examples, the average diameter of the droplets may be less than about 1 mm, less than about 500 micrometers, less than about 300 micrometers, less than about 200 micrometers, less than about 100 micrometers, less than about 75 micrometers, less than about 50 micrometers, less than about 30 micrometers, less than about 25 micrometers, less than about 20 micrometers, less than about 15 micrometers, less than about 10 micrometers, less than about 5 micrometers, less than about 3 micrometers, less than about 2 micrometers, less than about 1 micrometer, less than about 500 nm, less than about 300 nm, less than about 100 nm, or less than about 50 nm. The average diameter of the droplets may also be at least about 30 nm, at least about 50 nm, at least about 100 nm, at least about 300 nm, at least about 500 nm, at least about 1 micrometer, at least about 2 micrometers, at least about 3 micrometers, at least about 5 micrometers, at least about 10 micrometers, at least about 15 micrometers, or at least about 20 micrometers in certain cases. The “average diameter” of a population of droplets is the arithmetic average of the diameters of the droplets.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., the droplets may have a distribution of diameters such that no more than about 5%, no more than about 2%, or no more than about 1% of the droplets have a diameter less than about 90% (or less than about 95%, or less than about 99%) and/or greater than about 110% (or greater than about 105%, or greater than about 101%) of the overall average diameter of the plurality of droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link et al., published as WO 2004/091763 on Oct. 28, 2004, incorporated herein by reference.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Certain embodiments are generally directed to systems and methods for splitting a droplet into two or more droplets. For example, a droplet can be split using an applied electric field. The droplet may have a greater electrical conductivity than the surrounding liquid, and, in some cases, the droplet may be neutrally charged. In certain embodiments, in an applied electric field, electric charge may be urged to migrate from the interior of the droplet to the surface to be distributed thereon, which may thereby cancel the electric field experienced in the interior of the droplet. In some embodiments, the electric charge on the surface of the droplet may also experience a force due to the applied electric field, which causes charges having opposite polarities to migrate in opposite directions. The charge migration may, in some cases, cause the drop to be pulled apart into two separate droplets.

Some embodiments of the invention generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

Yet another aspect of the present invention is generally directed to kits, e.g., for amplifying or cloning within droplets. In some embodiments, the kit may include one or more components selected so as to facilitate the performance of one or more methods described herein. For instance, the kit may include a package or an assembly including one or more components such as those discussed herein. Other components may also be included within the kit, e.g., packaging or protective materials, assorted equipment such as beakers, flasks, vials, pipettes, microwell plates, collection tubes, instructions, or the like.

In certain embodiments, the kit may include a plurality of droplets, e.g., contained within a suitable container such as a tube, for example, for use as second microfluidic droplets that are free of a species of interest. Droplets such as second microfluidic droplets have been described herein, including concentrations or amounts. In some cases, the droplets may be formed from oils and/or surfactants (including those described in detail herein). In addition, the second microfluidic droplets may have substantially (or exactly) the same compositions, or different compositions, e.g., as previously discussed. The droplets may also be contained within a suitable aqueous or hydrophilic liquid, e.g., water and other aqueous solutions comprising water, such as cell or biological media, ethanol, salt solutions, etc. The kit may have any suitable volume of second microfluidic droplets, e.g., contained within a suitable liquid, such as an aqueous or hydrophilic liquid. For instance, the kit may have at least about 1 ml, at least about 2 ml, at least about 3 ml, at least about 5 ml, at least about 7 ml, at least about 10 ml, at least about 20 ml, at least about 30 ml, at least about 50 ml, at least about 100 ml, etc. of liquid containing droplets.

The kit, in certain embodiments, may include suitable hydrophobic liquids and/or surfactants. In certain embodiments, the hydrophobic liquid is one that is substantially immiscible in water, e.g., under ambient temperature and pressure. In some cases, the liquids are contained within a suitable container such as a tube. Non-limiting examples of hydrophobic liquids include oils such as hydrocarbons, silicon oils, fluorocarbon oils, organic solvents etc. Examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. Non-limiting examples of surfactants include those discussed in U.S. Pat. Apl. Pub. No. 2010/0105112, incorporated herein by reference. Other non-limiting examples of surfactants include Span80 (Sigma), Span80/Tween-20 (Sigma), Span80/Triton X-100 (Sigma), Abil EM90 (Degussa), Abil we09 (Degussa), polyglycerol polyricinoleate “PGPR90” (Danisco), Tween-85, 749 Fluid (Dow Corning), the ammonium carboxylate salt of Krytox 157 FSL (Dupont), the ammonium carboxylate salt of Krytox 157 FSM (Dupont), or the ammonium carboxylate salt of Krytox 157 FSH (Dupont).

In some embodiments, the kit may also include a signaling entity, e.g., which can be added to cells, droplets or the like. The signaling entity may be fluorescent in some cases. As other non-limiting examples, the signaling entity may be a dye such as fluorescent dye, a radioactive atom or compound, etc. The signaling entity may also be an ultraviolet dye or an infrared dye in some cases. Examples of signaling entities include, but are not limited to, calcein (or calcein derivatives such calcein AM), propidium iodide, 7-aminoactinomycin D, nuclear stains, Calcein Blue AM, Calcein Violet AM, Fura-2 AM, Indo-1 AM, resazurin, and the like. Many such dyes are commercially available. Determination of the signaling entity may occur using techniques such as radioactivity, fluorescence, phosphorescence, light scattering, light absorption, fluorescence polarization, or the like.

In certain embodiments, the kit may also include a cell-counting device for counting droplets. For example, the kit may include a hemocytometer or a glass capillary. Many such counting chambers are commercially available.

In some embodiments, the kit may include instructions in any form that are provided in connection with the kit. For instance, the instructions may include instructions for the use, modification, mixing, diluting, preserving, administering, assembly, storage, packaging, and/or preparation of the components associated with the kit. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

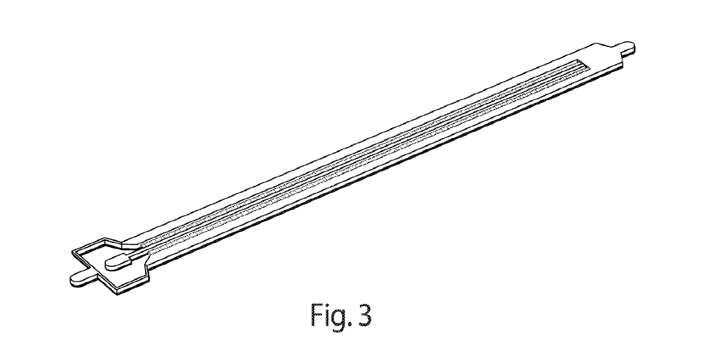

In certain embodiments, the kit may include a device for making droplets, e.g., microfluidic droplets. One non-limiting example, of such a droplet-making device is discussed below and with reference to FIG. 3; however, other droplet-making devices are also possible, including those known to those of ordinary skill in the art. In FIG. 3, the apparatus comprises a first channel, a second channel, and a plurality of side channels each connecting the first channel with the second channel. Some or all of these channels may be microfluidic. A first fluid may enter through a first channel while a second fluid enters through a second channel. The first fluid can flow through the side channels to enter the second channel. If the first fluid and the second fluid are at least substantially immiscible, the first fluid exiting the side channels may form individual droplets within the second channel, as is shown by the droplets. In addition, in certain embodiments, the first fluid itself may contain an emulsion. Additional details may be seen in Int. Pat. Apl. Pub. No. PCT/US2014/037962, filed May 14, 2014, entitled “Rapid Production of Droplets,” by Weitz, et al., incorporated herein by reference in its entirety.

The side channels, in some cases, may each have substantially the same dimensions, e.g., they may have substantially the same volume, cross-sectional area, length, shape, etc. For example, each of the first channel and the second channel may be substantially straight and parallel, and/or the first and second channels may not necessarily be straight but the channels may have a relatively constant distance of separation therebetween, such that some or all of the side channels have substantially the same shape or other dimensions while connecting the first channel with the second channel.

As mentioned, fluid passing from the first channel through the side channels, and entering the second channel, may form a plurality of droplets of first fluid contained within the second fluid. In some cases, the droplets may have substantially the same size or characteristic dimension, for example, if the side channels have substantially the same cross-sectional area and/or length and/or other dimensions. In such a way, a plurality of substantially monodisperse droplets may be formed, in accordance with certain embodiments of the invention.

However, although the side channels are shown in FIG. 3 are shown as being straight, with constant cross-sectional area, this is by way of example only, and in other embodiments, the side channels need not be straight, and/or the side channels may not necessarily have a constant cross-sectional area. For example, the side channels may have different cross-sectional areas at different locations within the channels. In addition, other channels may be present in connection with these channels in certain embodiments. Furthermore, although the side channels are illustrated as being regularly periodically spaced in FIG. 3, this is not a requirement, and other spacings of the side channels are also possible in other cases. For example, in one set of embodiments, the spacings between adjacent channels may be substantially the same, and/or the cross-sectional dimension or area of the side channels may be substantially the same size to create droplets that have substantially the same size or average diameter.

In one set of embodiments, the minimum cross-sectional area of the side channels is substantially smaller than the cross-sectional area of the first or second channels. For example, the first channel may have a cross-sectional area at least 10 times larger than the smallest cross-sectional area of the side channels. In some cases, the height of the first channel and the height of the side channels may be different, e.g., to produce such differences in cross-sectional area. Without wishing to be bound by any theory, it is believed that since the cross-sectional area of the side channels is substantially smaller than the cross-sectional area of the first or second channels, the resistance to fluid flow is largely dominated by the dimensions of the side channels, rather than the dimensions of the first or second channels. Accordingly, if the side channels have substantially the same dimensions, the side channels should each produce substantially the same resistance to fluid flow, and accordingly, produce droplets are substantially the same. Thus, by controlling factors such as the overall pressure drop across the side channels to be substantially constant, a plurality of substantially monodisperse droplets may be produced, at least according to some embodiments of the invention.

It should also be understood that the first channel and the second channel may be of any suitable length. In some embodiments, relatively long channels may be used, e.g., such that a relatively large number of side channels may be present between the first and second channels, which may be used to produce relatively large numbers of droplets and/or to produce droplets at relatively large rates. For example, there may be at least 100, 500, 1,000, etc. side channels present between the first channel and the second channel. In addition, in certain embodiments, the first and/or second channels may have a length of at least 1 mm, at least 5 mm, at least 1 cm, at least 2 cm, at least 3 cm, etc.

A variety of materials and methods, according to certain aspects of the invention, can be used to form certain articles or components such as those described herein, e.g., channels such as microfluidic channels, chambers, microfluidic devices (e.g., for creating droplets, manipulating droplets, causing amplification within droplets, etc.), or the like. For example, various articles or components can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al).

In one set of embodiments, various structures or components of the articles described herein can be formed of a polymer, for example, an elastomeric polymer such as polydimethylsiloxane (“PDMS”), polytetrafluoroethylene (“PTFE” or Teflon®), or the like. For instance, according to one embodiment, a microfluidic channel may be implemented by fabricating the fluidic system separately using PDMS or other soft lithography techniques (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference).

Other examples of potentially suitable polymers include, but are not limited to, polyethylene terephthalate (PET), polyacrylate, polymethacrylate, polycarbonate, polystyrene, polyethylene, polypropylene, polyvinylchloride, cyclic olefin copolymer (COC), polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The device may also be formed from composite materials, for example, a composite of a polymer and a semiconductor material.

In some embodiments, various structures or components of the article are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, waxes, metals, or mixtures or composites thereof heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, dodecyltrichlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of various structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures or channels from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, structures can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Thus, in certain embodiments, the design and/or fabrication of the article may be relatively simple, e.g., by using relatively well-known soft lithography and other techniques such as those described herein. In addition, in some embodiments, rapid and/or customized design of the article is possible, for example, in terms of geometry. In one set of embodiments, the article may be produced to be disposable, for example, in embodiments where the article is used with substances that are radioactive, toxic, poisonous, reactive, biohazardous, etc., and/or where the profile of the substance (e.g., the toxicology profile, the radioactivity profile, etc.) is unknown. Another advantage to forming channels or other structures (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

The following documents are incorporated herein by reference in their entirety for all purposes: Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; Int. Pat. Apl. Pub. No. WO 2010/151776, entitled “Fluid Injection,” by Weitz, et al.; U.S. Pat. Apl. Ser. No. 61/981,123, entitled “Systems and Methods for Droplet Tagging,” by Bernstein, et al.; U.S. Pat. Apl. Ser. No. 61/981,108, entitled “Methods and Systems for Droplet Tagging and Amplification,” by Weitz, et al.; and Int. Pat. Apl. Pub. No. PCT/US2014/037962, filed May 14, 2014, entitled “Rapid Production of Droplets,” by Weitz, et al. Also incorporated herein by reference in its entirety is U.S. Provisional Patent Application Ser. No. 62/106,981, filed Jan. 23, 2015, entitled “Systems, Methods, and Kits for Amplifying or Cloning Within Droplets,” by Weitz, et al.

The following examples are intended to illustrate certain embodiments of the present invention, but do not exemplify the full scope of the invention.

### Example 1

Molecular cloning provides scientists with an essentially unlimited quantity of individual DNA segments derived from an original resource. However, because of its tedious procedures, low throughput, and the challenge for amplifying DNA to be cloned from rare templates, cloning is a rate-determining step (RDS) for many relevant biological studies. This example uses drop-based microfluidic digital PCR to expand single target molecules in individual pico-reactors, which mimics the process of colony formation and expansion from one successfully transfected competent cell. After isolating each positive drop, the numerous amplicons inside allows the efficient re-amplification to achieve enough materials for characterization, for example, by Sanger sequencing. Compared with conventional 4 days of continuous bench-work, amplification can occur in much reduced times, e.g., around 7 hours, and the dramatically increased throughput allows screening of, e.g., 300,000 reactions in a single experiment. This example demonstrates one sensitive, simple, and cost-effective approach for high-throughput molecular cloning.

Molecular cloning is a set of experimental methods in molecular biology that are used to assemble DNA molecules and to direct their expansion within host organisms, which can be used for a wide range of purposes, such as variant detection, genome organization, and gene expression. In standard molecular cloning experiments, the cloning of DNA fragment typically involves these steps: (1) choice of host organism and cloning vector; (2) preparation of vector DNA; (3) preparation of DNA to be cloned; (4) ligation of cloning vector and target DNA; (5) introduction into host organism; (6) screening for clones with desired DNA inserts and biological properties by means of PCR, restriction fragment analysis, and DNA sequencing. The whole procedure is time-consuming, labor-intensive, and low-throughput. In addition, when the target biological events are very rare, the acquisition of amplicon to be cloned is very challenging. More importantly, the preparation of DNA, usually PCR, almost always introduces bias and error, which cannot faithfully represent the genomic information in the original biological samples. Thus, the ability to fast clone and reliably characterize single molecules would significantly simplify and accelerate the molecular cloning.

Instead of increasing the signal-to-noise ratio by taking extra steps to reduce noise, an alternative method to overcome this low-sample-number limitation in the initial amplification is through the use of digital PCR (dPCR), in which single templates are compartmentalized into picoliter volumes, thereby decreasing the number of non-specific templates in compartments that contain a target template, and lowering the probability of non-specific amplification. A 1 mL blood sample containing 50 HIV particles and 10⁷ white blood cells would have a signal-to-noise ratio in bulk of 1:10⁶. After being compartmentalized into 1 nL droplets, a few droplets containing 1 virus at a concentration equivalent to 2×10⁵ cells per mL, and 10 white blood cells at 10⁷ cells per mL. These droplets will have a signal-to-noise ratio of 1:10, and thus will be easily distinguished from the vast majority of the droplets that contain no viruses and thus a much lower signal-to-noise ratio. Moreover, increasing the effective concentration of the signal will also improve the singal-to-PCR inhibitor ratio. Droplets are thus less susceptible to inhibitor concentrations that would obstruct PCR in bulk. This is especially relevant for, for example, blood, urea, and feces, which often have high concentrations of PCR inhibitors.

This example presents a drop-based microfluidic digital RT-PCR protocol that was used to amplify and characterize single recombinant RNA viruses that drive viral evolution. RNA viruses are among the most rapidly evolving organisms on earth, which enables them to escape immune systems, resist treatments, or switch between hosts. Recombination between two other closely related yet distinct viruses is one of the viral evolution processes. Although rare, it is often the cause of new and more virulent strains appearing, while mutations in viral genes change the virus slowly because of the gradually accumulation over time. At the time of recombination, each of the rare viruses contains unique and valuable information on the newly emerging disease. To identify and isolate these recombinants as early as possible, it is often necessary to selectively amplify rare RNA genomes in a background of many other RNA genomes. Moreover, since each mutant carries unique information, it must be sequenced individually. The protocol used in this example uses drop-based microfluidic digital RT-PCR to enhance dPCR to make it applicable to single recombinant RNA molecules and to recover the product would be of tremendous value in identifying rare variants in viral diseases. The system may also be used with commercially available Taqman probes, as well as experimenter-designed primers in conjunction with DNA-binding dyes such as EvaGreen.

This example uses drop-based microfluidic digital PCR for single viral genome amplification, allowing the investigation of, for example, 300,000 reactions in a single experiment. Although the example described below is specific to viral stock, this protocol could also be applied to any kind of DNA or RNA fragment, for example, in the context of investigating gene mutations which relates with drug resistance and cancer heterogeneity. Molecular cloning, using the protocol described here allows sensitive, simple, cost-effective, and high-throughput molecular cloning. This would also be of great value to other applications such as the study of the regulation of biological functions in rare cells, especially in cancer.

This example involves compartmentalization of single DNA and/or RNA molecules into separate droplets, their amplification, quantification, and/or sequencing. This example shows a procedure to retrieve amplified DNA/RNA molecules in droplets and sequence them individually. This can be described through set of components (kit) to perform “cloning-in-drop.” This example comprises the following principal steps:
i) Template encapsulation into droplets;
ii) Vector-free gene amplification in drops (for clonal replication of templates);
iii) Droplet quantification with hemocytometer, glass slide or other techniques;
iv) Distribution of single PCR positive drops into individual wells or containers by diluting them with empty droplets; the latter droplets may carry PCR reagents, primers, enzymes and other biochemicals necessary for second round amplification; and
v) Second-round amplification to acquire materials for sequencing, e.g., Sanger sequencing.

These steps can be finished in, for example, a couple of hours, while regular bacterial cell based cloning and sequencing will often take more than two days to complete. Therefore, this example is very fast, which allows, for instance, the analysis of a large number of target templates at single-molecule level. By performing template pre-amplification in droplets following dispensing droplets into individual wells, the example provides a new approach to improve detection of single DNA/RNA molecules in the sample using, for example, 96-, 384-, or 1536-microwell plates. Without encapsulation and pre-amplification steps (i-ii), quantification of DNA/RNA templates in a sample could be hindered by PCR biases, poor amplification efficiency in larger volume or possible contamination (multiple templates present in a single well).

In some cases, this example, allows for single templates to be isolated from a mixed population, which allows single-nucleic acid template as well as single-cell isolation from the sample. This allows, for example, unbiased amplification and accurate quantification of individual template molecules in the original sample; separation of merged genetic materials from samples containing difficult-to-dissociate cells, such as tumor, embedded samples and brain tissues; increased relative concentrations of a template and detection of rare molecules; increased sensitivity and lower template detection thresholds; and avoidance of interference of chimera and stutter amplicons. Other advantages include single genome amplification and the investigation of gene mutations relating to drug resistance or cancer heterogeneity.

Especially, in case the biological events are very rare, the acquisition of target DNA fragment to be cloned in an excess of background is very challenging, because polymerase chain reaction (PCR) method, which is often used for amplification of specific DNA or RNA (RT-PCR) sequences prior to molecular cloning, does not always work. For standard volumes and preparation methods, target templates are amplified from an initial concentration of 10⁶ molecules per mL. This fairly high template concentration is necessary because PCR may amplify non-specific DNA that does not match the target sequence, especially under sub-optimal conditions. The concentration of specific template, or signal, must be sufficiently high to overcome this noise. However, clinical samples such as blood from acquired immune deficiency syndrome (AIDS) patients experiencing low-level replication may carry human immunodeficiency virus (HIV) particles at concentrations <50 copies per mL, against a background of 10⁷ white blood cells per mL. Even if the white blood cells are somehow removed, the reaction will easily be contaminated by aerosols, dust, and other incidental sources of noise. Careful and specialized sample preparation is therefore necessary to eliminate extraneous DNA and minimize noise.

### Example 2

High-throughput screening (HTS) is a method for drug discovery. Using robotics, data processing and control software, liquid handling devices, and sensitive detectors, a researcher can conduct large scale of pharmacological tests, or identify active compounds, antibodies, or genes that modulate a particular biomolecular pathway. The key labware of HTS is the microtiter plate, which can have, e.g., 384, 1536, or 3456 wells, and current robots can often test up to 100,000 compounds per day. However, this technology is approaching its physical limit; below the 1-microliter-volumes of 1,536-well plates, evaporation and capillary forces become significant. Developments on microwell-based microfluidic technology have significantly improved screening capabilities, increased the speed by 10-fold and decreased the reaction volume by 1,000-fold. The use of water-in-oil drops eliminates solid wells used in microtiter plates; this can simplify engineering and/or expand the capacity of drug screening within an acceptable time and cost scale. This is demonstrated in this example, where droplets are multi-functionalizd to demonstrate drug screening with a high level of combinations, e.g., to test their synergistic effects on cells.

To construct massive drug combinations, three groups of relatively monodisperse picoliter drops were first individually generated in 96-parallel microfluidic drop-makers. Each group included 96 kinds of drops with different drugs and their different concentrations, along with a unique pre-mixed oligonucleotide index in the solution (96 was used here as an illustrative example, although other numbers of drops could have been used in other embodiments). These three groups of drops were then merged using a microfluidic drop-merger in a random combination of different drugs and different concentrations. Single K562 chronic myeloid leukemia cells were introduced to the drug combinations by picoinjecting a cell suspension to the merged drops at a concentration known to obtain a Poisson distribution with rate λ (lambda)=0.1, and incubated at 37° C. for 24 hours. See, e.g., U.S. Pat. Apl. Pub. No. 2012/0132288, entitled “Fluid Injection,” incorporated herein by reference in its entirety.

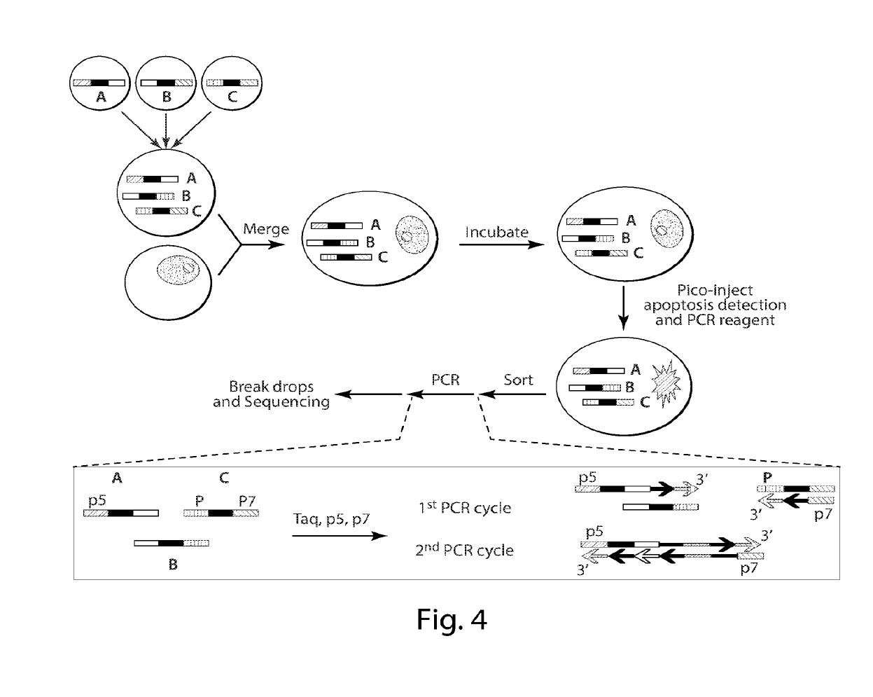

By adding a fluorogenic substrate, caspalux6-J1D2, which is specifically cleaved by increased caspase 3 and caspase 3-like activities during apoptosis, apoptosis of cells in drops can be determined. In this apoptosis assay solution, a PCR cocktail was included to link the oligonucleotide indexes to a full-length double-stranded DNA barcode through PCR amplification. After incubation at 37° C. for half an hour, the drops containing apoptotic cells that suggest effective drug combinations were sorted according to fluorescence intensity, followed by PCR amplification and next-generation sequencing (NGS) to decode the double-stranded DNA barcodes in each sorted drops, which were used to reveal the optimal drug combinations. The schema of this large-scale drug combination screening system is shown in FIG. 4, showing large-scale drug combination screening in drop-based microfluidics.

The strategy to create a double-stranded DNA “barcode” representing three oligos/three drugs and their different concentrations is presented in the FIG. 4 inset. To form this DNA barcode, three families of oligonucleotide indexes are used, a left oligonucleotide (A), a center oligonucleotide (B) and a right oligonucleotide (C). The left (A) and center (B) partially overlap, and the center (B) and right (C) partially overlap. These overlaps allow the three oligonucleotides to anneal to each other when they are present in a single drop, as discussed below. The drug defining unique barcode is encoded in the non-overlapping parts of the left, center and right oligonucleotides. After two rounds of PCR, these three oligonucleotides result in a double stranded “ABC” DNA “barcode.” To allow the DNA barcode to be sequenced through NGS, common sequencing primers P5 and P7 are integrated on the 5′ end of the left (A) oligonucleotide and the 3′ end of the right (C) oligonucleotide, respectively. The annealing and PCR are performed within individual droplets, e.g., to make sure the 3 barcodes are linked together to allow subsequent sequence analysis to reveal what 3 drugs were combined based on the oligonucleotides within the “barcode.” A bioinformatics pipeline to decode the DNA barcodes from NGS reads has been developed.

An even annealing and amplification of combinations of four A, four B and four C oligos in bulk, 64 barcode combinations in total, is shown in FIG. 5; this property allows for quantitatively analyzing how many cells have been induced to undergo apoptosis by counting the unique barcode reads. Furthermore, another advantage for this drop-based platform to perform quantitative apoptosis detection is that the loss of apoptotic cells was minimized compared with bulk assays, in which several staining and washing steps diminish the accuracy for apoptosis detection.

FIG. 5 shows even amplification of 64 barcode combinations in bulk decoded by deep sequencing, representing three oligonucleotides/three drugs and their different concentration combinations. It should be noted that each “barcode” was amplified by substantially the same amount, i.e., the amplification was “even,” e.g., rather than favoring one or two barcodes at the expense of the other barcodes.

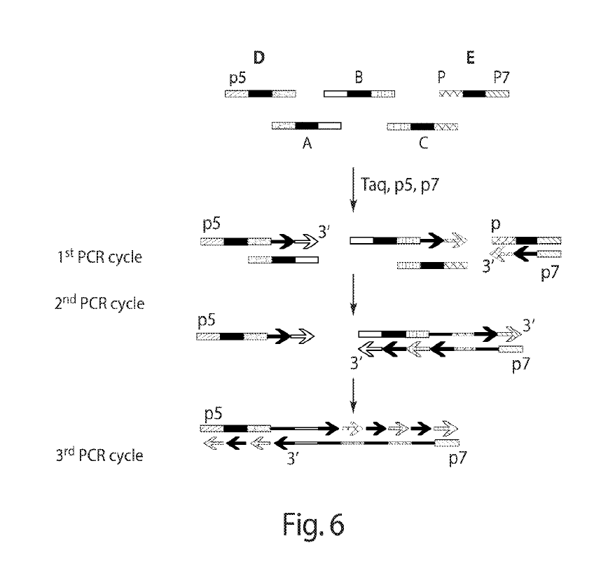

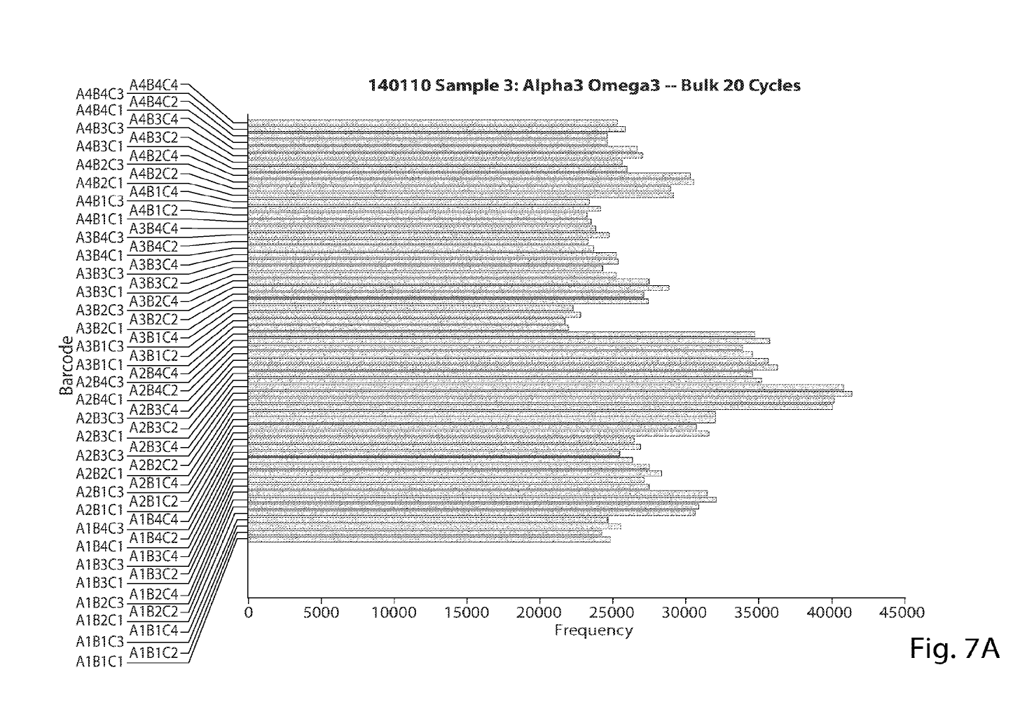

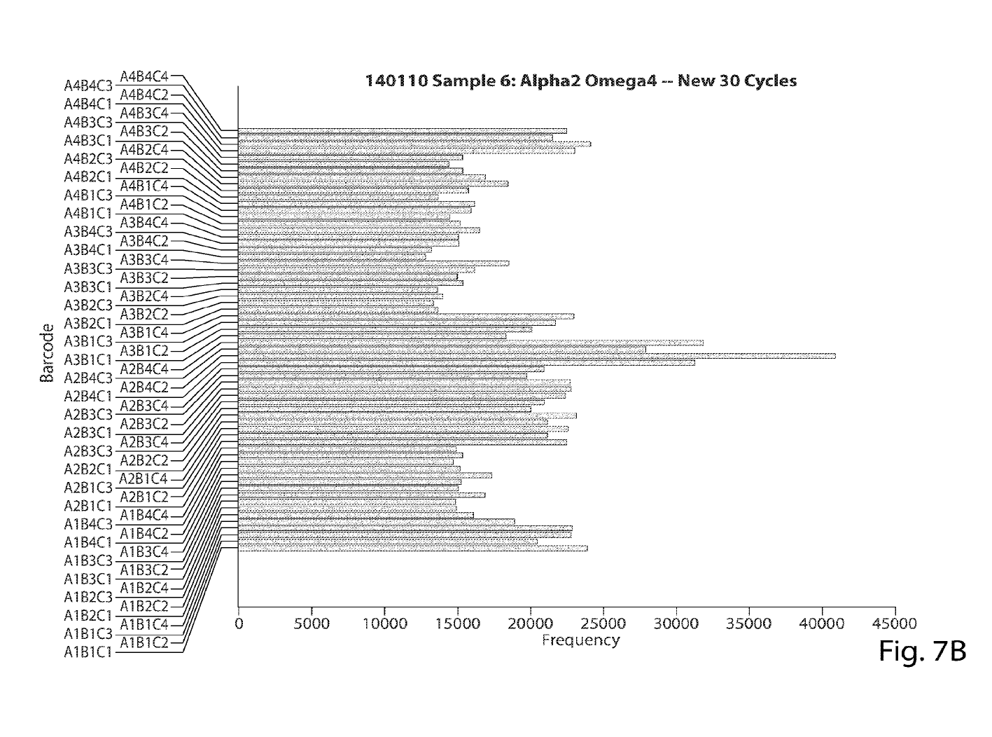

Given that each group of drops had at most 96 kinds of drops with different drugs and their different concentrations, and there are a total of three groups of drops, nearly 1 million drug combinations could be obtained (96×96×96). To further scale up the screening in terms of multiple cell lines without increasing the deep-sequencing run, two oligonucleotide indexes D and E were added into the solution containing the PCR cocktail in another set of experiments. The formation of double stranded DNA barcodes shared a similar mechanism as that described above. Briefly, the newly added oligo indexes D and E integrate P5 had P7 sequences, and partially overlap with oligo A and C, respectively. Instead of in two cycles of PCR, the final barcode DNA was constructed in three cycles of PCR, as shown in FIG. 6, showing a strategy to create a double-stranded DNA barcode combining three oligonucleotide tagging drugs and two more barcodes tagging the cell lines. Even amplifications of 64 barcode combinations with two more barcodes to tag the cell lines is shown in both bulk and drop-based amplification (FIG. 7). This strategy allowed screening of drug combinations and cell lines in a high-throughput and cost- and time-effective way in this example.

FIG. 7 shows even amplification of 64 barcode combinations with 2 more barcodes to tag the samples is shown by deep sequencing. FIG. 7A shows bulk amplification, while FIG. 7B shows drop-based amplification.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
fragmenting a nucleic acid to produce nucleic acid fragments;
containing at least some of the nucleic acid fragments in a plurality of microfluidic droplets; and
amplifying at least some of the nucleic acid fragments contained within the microfluidic droplets.

2. The method of claim 1, comprising using PCR to amplifying at least some of the nucleic acid fragments contained within the microfluidic droplets.

3. The method of any one of claim 1 or 2, wherein amplifying at least some of the nucleic acid fragments comprises adding a polymerase to at least some of the microfluidic droplets.

4. The method of claim 3, wherein the polymerase is DNA polymerase.

5. The method of claim 3, wherein the polymerase is RNA polymerase.

6. The method of any one of claims 1-5, wherein amplifying at least some of the nucleic acid fragments comprises adding Taq polymerase to at least some of the microfluidic droplets.

7. The method of any one of claims 1-6, wherein at least some of the microfluidic droplets comprise Taq polymerase.

8. The method of any one of claims 1-7, comprising exposing at least some of the microfluidic droplets to a temperature of at least about 50° C.

9. The method of any one of claims 1-8, comprising exposing at least some of the microfluidic droplets to a temperature of at least about 90° C.

10. The method of any one of claims 1-9, wherein amplifying at least some of the nucleic acid fragments comprises adding deoxyribonucleotides to at least some of the microfluidic droplets.

11. The method of any one of claims 1-10, wherein at least some of the microfluidic droplets comprise deoxyribonucleotides.

12. The method of any one of claims 1-11, wherein the nucleic acid is DNA.

13. The method of any one of claims 1-12, wherein the nucleic acid comprises genomic DNA.

14. The method of any one of claims 1-13, wherein the nucleic acid comprises bacterial DNA.

15. The method of any one of claims 1-13, wherein the nucleic acid comprises virial DNA.

16. The method of any one of claims 1-11, wherein the nucleic acid comprises RNA.

17. The method of any one of claims 1-11, wherein the nucleic acid comprises virial RNA.

18. The method of any one of claims 1-17, wherein the nucleic acid comprises nucleic acid arising from a cell.

19. The method of claim 18, wherein the cell is a human cell.

20. The method of any one of claim 18 or 19, wherein the cell is a cancer cell.

21. The method of any one of claims 1-20, wherein the nucleic acid comprises nucleic acid arising from more than one cell.

22. The method of any one of claims 1-21, wherein the nucleic acid comprises nucleic acid arising from more than one organism.

23. The method of any one of claims 1-22, wherein the nucleic acid comprises nucleic acid arising from more than one species.

24. The method of any one of claims 1-23, wherein the plurality of microfluidic droplets have an average diameter of less than about 1 mm.

25. The method of any one of claims 1-24, wherein the plurality of microfluidic droplets have a distribution of diameters such that no more than about 5% of the microfluidic droplets have a diameter less than about 90% or greater than about 110% of the overall average diameter of the microfluidic droplets.

26. The method of any one of claims 1-25, further comprising mixing the plurality of microfluidic droplets with a second plurality of microfluidic droplets.

27. The method of any one of claims 1-26, further comprising mixing the microfluidic droplets with second microfluidic droplets free of the nucleic acid fragments to produce a mixture of microfluidic droplets.

28. The method of claim 27, comprising mixing the microfluidic droplets with the second microfluidic droplets at a ratio of at least 1:1,000.

29. The method of any one of claims 1-28, further comprising transferring at least some of the microfluidic droplets into a container.

30. The method of claim 29, further comprising amplifying nucleic acid within the microfluidic droplets transferred into the container.

31. The method of any one of claims 1-30, comprising pipetting at least some of the microfluidic droplets into a container.

32. The method of any one of claims 1-31, further comprising bursting at least some of the microfluidic droplets.

33. The method of any one of claims 1-32, further comprising sequencing the nucleic acid fragments.

34. The method of any one of claims 1-33, further comprising attaching a nucleic acid barcode to at least some of the nucleic acid fragments contained in the plurality of microfluidic droplets.

35. The method of any one of claims 1-31, further comprising disrupting the plurality of microfluidic droplets and collecting the amplified nucleic acid in a common solution.

36. The method of claim 35, further comprising containing the amplified nucleic acid in the common solution in a third plurality of microfluidic droplets.

37. The method of claim 36, further comprising amplifying at least some of the amplified nucleic acids within the second plurality of microfluidic droplets.

38. The method of any one of claim 36 or 37, further comprising attaching a nucleic acid barcode to at least some of the amplified nucleic acids contained in the second plurality of microfluidic droplets.

39. A method, comprising:
containing nucleic acid in a plurality of microfluidic droplets; and
evenly amplifying at least some of the nucleic acid contained within the microfluidic droplets.

40. A method, comprising:
evenly amplifying a plurality of nucleic acids contained within microfluidic droplets.

41. A method, comprising:
mixing first microfluidic droplets containing a species of interest with second microfluidic droplets free of the species of interest to produce a mixture of microfluidic droplets; and
transferring at least 10 nl of the mixture of microfluidic droplets into a container.

42. The method of claim 41 comprising transferring, on average, only one first microfluidic droplet into the container.

43. The method of any one of claim 41 or 42, wherein the ratio of first microfluidic droplets to second microfluidic droplets is at least about 1:1,000.

44. The method of any one of claims 41-43, wherein the ratio of first microfluidic droplets to second microfluidic droplets is at least about 1:10,000.

45. The method of any one of claims 41-44, wherein the ratio of first microfluidic droplets to second microfluidic droplets is at least about 1:100,000.

46. The method of any one of claims 41-45, wherein the first microfluidic droplets have an average diameter of no more than about 1 micrometer.

47. The method of any one of claims 41-46, wherein the second microfluidic droplets have an average diameter of no more than about 1 micrometer.

48. The method of any one of claims 41-47, wherein the first microfluidic droplets each have an average diameter of between about 90% and about 110% of the average diameter of the first microfluidic droplets.

49. The method of any one of claims 41-48, wherein the second microfluidic droplets each have an average diameter of between about 90% and about 110% of the average diameter of the second microfluidic droplets.

50. The method of any one of claims 41-49, wherein the second microfluidic droplets have substantially the same composition.

51. The method of any one of claims 41-50, wherein the species of interest is a nucleic acid.

52. The method of any one of claims 41-51, wherein the species of interest is DNA.

53. The method of any one of claims 41-52, wherein the species of interest is genomic DNA.

54. The method of any one of claims 41-53, wherein the container is a well of a microwell plate.

55. The method of claim 54, wherein the microwell plate is a 96-well microwell plate.

56. The method of any one of claims 41-55, comprising transferring at least about 100 nl of the mixture of microfluidic droplets into a container.

57. The method of any one of claims 41-56, comprising transferring at least about 1 microliter of the mixture of microfluidic droplets into a container.

58. The method of any one of claims 41-57, wherein transferring comprises pipetting.

59. The method of claim 58, comprising automatically pipetting the mixture of microfluidic droplets into a container.

60. The method of claim 58, comprising manually pipetting the mixture of microfluidic droplets into a container.

61. The method of any one of claims 41-60, wherein the mixture of microfluidic droplets has a total volume of microfluidic droplets of at least about 1 microliter.

62. The method of any one of claims 41-61, wherein the mixture of microfluidic droplets has a total volume of microfluidic droplets of at least about 10 microliters.

63. The method of any one of claims 41-62, wherein the mixture of microfluidic droplets has a total volume of microfluidic droplets of at least about 100 microliters.

64. The method of any one of claims 41-63, wherein the mixture of microfluidic droplets has a total volume of microfluidic droplets of at least about 1 ml.

65. The method of any one of claims 41-64, wherein the second microfluidic droplets have an average diameter of less than about 1 mm.

66. A method, comprising:
mixing first microfluidic droplets containing a species of interest with second microfluidic droplets free of the species of interest to produce a mixed fluid containing the microfluidic droplets to produce a mixture of microfluidic droplets; and
transferring, on average, a plurality of second microfluidic droplets and no more than about 1.5 first microfluidic droplets into a container.

67. A kit, comprising:
a droplet-making device configured to produce microfluidic droplets;
a microfluidic device configured to manipulate the microfluidic droplets; and
a container containing a plurality of microfluidic droplets having substantially the same composition.

68. The kit of claim 67, further comprising a second container containing a fluid substantially immiscible in water.

69. The kit of any one of claim 67 or 68, further comprising a fluorescent dye.

70. The kit of any one of claims 67-69, further comprising a cell-counting device.

71. The kit of any one of claims 67-70, wherein the droplet-making device comprises a first microfluidic channel, a second microfluidic channel, and at least five side microfluidic channels each connecting the first microfluidic channel with the second microfluidic channel, wherein the first microfluidic channel has a cross-sectional area at least 20 times greater than the smallest cross-sectional area of the at least five side channels.

72. The kit of any one of claims 67-71, wherein the droplet-making device comprises a first, microfluidic channel having a length of at least about 5 mm, a second microfluidic channel substantially parallel to the first microfluidic channel, and at least five side microfluidic channels each connecting the first microfluidic channel with the second microfluidic channel.

73. The kit of any one of claims 67-72, wherein the droplet-making device comprises a first microfluidic channel having a length of at least about 5 mm, a second microfluidic channel, at least five side microfluidic channels each connecting the first microfluidic channel with the second microfluidic channel, a third microfluidic channel, and at least five side microfluidic channels each connecting the second microfluidic channel with the third microfluidic channel.

74. The kit of any one of claims 67-73, wherein the droplet-making device comprises a first microfluidic channel, a second microfluidic channel, at least five side microfluidic channels each connecting the first microfluidic channel with the second microfluidic channel, and a plurality of auxiliary microfluidic channels connecting to each of the at least five side microfluidic channels.

75. The kit of any one of claims 67-74, wherein the microfluidic device is configured to fuse microfluidic droplets with a fluid containing a nucleic acid.

76. A method, comprising:
containing a plurality of nucleic acids in a first plurality of microfluidic droplets;
amplifying at least some of the nucleic acids within the first plurality of microfluidic droplets;
combining the amplified nucleic acids in a common solution;
containing the amplified nucleic acids in a second plurality of microfluidic droplets; and
amplifying at least some of the amplified nucleic acids within the second plurality of microfluidic droplets.

77. The method of claim 76 comprising using PCR to amplify at least some of the nucleic acids within the first plurality of microfluidic droplets.

78. The method of claim 77 wherein amplifying at least some of the nucleic acids within the first plurality of microfluidic droplets comprises adding a polymerase to at least some of the first plurality of microfluidic droplets.

79. The method of claim 78, wherein the polymerase is DNA polymerase.

80. The method of claim 78, wherein the polymerase is RNA polymerase.

81. The method of any one of claims 76-80, wherein amplifying at least some of the nucleic acids within the first plurality of microfluidic droplets comprises adding Taq polymerase to at least some of the first plurality of microfluidic droplets.

82. The method of any one of claims 76-81, wherein at least some of the first plurality of microfluidic droplets comprise Taq polymerase.

83. The method of any one of claims 76-82, comprising exposing at least some of the first plurality of microfluidic droplets to a temperature of at least about 50° C.

84. The method of any one of claims 76-83, comprising exposing at least some of the first plurality of microfluidic droplets to a temperature of at least about 90° C.

85. The method of any one of claims 76-84, wherein amplifying at least some of the nucleic acids within the first plurality of microfluidic droplets comprises adding deoxyribonucleotides to at least some of the first plurality of microfluidic droplets.

86. The method of any one of claims 76-85, wherein at least some of the first plurality of microfluidic droplets comprise deoxyribonucleotides.

87. The method of any one of claims 76-86, further comprising fragmenting a nucleic acid to produce the plurality of nucleic acids.

88. The method of any one of claims 76-87, wherein the plurality of nucleic acids comprises DNA.

89. The method of any one of claims 76-88, wherein the plurality of nucleic acids comprises RNA.

90. The method of any one of claims 76-89, wherein the plurality of nucleic acids comprises genomic DNA.

91. The method of any one of claims 76-90, wherein the plurality of nucleic acids comprises bacterial DNA.

92. The method of any one of claims 76-91, wherein the plurality of nucleic acids comprises virial DNA.

93. The method of any one of claims 76-92, wherein the plurality of nucleic acids comprises virial DNA.

94. The method of any one of claims 76-93, wherein the plurality of nucleic acids comprises nucleic acid arising from a cell.

95. The method of claim 94, wherein the cell is a human cell.

96. The method of any one of claim 94 or 95, wherein the cell is a cancer cell.

97. The method of any one of claims 76-96, wherein the plurality of nucleic acids comprises nucleic acid arising from more than one cell.

98. The method of any one of claims 76-97, wherein the plurality of nucleic acids comprises nucleic acid arising from more than one organism.

99. The method of any one of claims 76-98, wherein the plurality of nucleic acids comprises nucleic acid arising from more than one species.

100. The method of any one of claims 76-99, wherein the plurality of first microfluidic droplets have an average diameter of less than about 1 mm.

101. The method any one of claims 76-100, wherein the plurality of first microfluidic droplets have a distribution of diameters such that no more than about 5% of the microfluidic droplets have a diameter less than about 90% or greater than about 110% of the overall average diameter of the microfluidic droplets.

102. The method of any one of claims 76-101, wherein the plurality of second microfluidic droplets have an average diameter of less than about 1 mm.

103. The method of any one of claims 76-102, wherein the plurality of second microfluidic droplets have a distribution of diameters such that no more than about 5% of the microfluidic droplets have a diameter less than about 90% or greater than about 110% of the overall average diameter of the microfluidic droplets.

104. The method of any one of claims 76-103, wherein combining the amplified nucleic acids in a common solution comprises disrupting the droplets and collecting the amplified nucleic acids within the common solution.

105. The method of claim 104, wherein disrupting the droplets comprises exposing the droplets to ultrasound.

106. The method of any one of claim 104 or 105, wherein disrupting the droplets comprises exposing the droplets to mechanical disruption.

107. The method of any one of claims 76-106, wherein amplifying at least some of the amplified nucleic acids within the second plurality of microfluidic droplets comprises attaching a nucleic acid barcode to at least some of the amplified nucleic acids contained in the second plurality of microfluidic droplets.

108. The method of any one of claims 76-107, comprising using PCR to amplify at least some of the nucleic acids within the second plurality of microfluidic droplets.
